(19) United States
(12) Patent Application Publication
(10) Pub. No.: US 2009/0004121 A1
(43) Pub. Date: Jan. 1, 2009

(54) Combination of picotamide with nafronyl

(76) Inventor: Herman J. Kempen, Dornach (CH)

Correspondence Address: WENDEROTH, LIND & PONACK, L.L.P., 2033 K STREET N. W., SUITE 800, WASHINGTON, DC, 20006-1021, US

(21) Appl. No.: 12/216,029

(22) Filed: Jun. 27, 2008

(30) Foreign Application Priority Data
Jun. 28, 2007 (EP) 07111244.5

(51) Int. Cl. A61K 9/68; A61K 31/444; A61P 9/00

(52) U.S. Cl. 424/48; 514/332

(57) ABSTRACT

The invention relates to a combination of picotamide(N,N′-bis-(3-picolyl)-4-methoxy-isophthalamide) and nafronyl(2-diethylaminoethyl 2-(naphthalen-1-ylmethyl)-3-(oxolan-2-yl)propanoate), pharmaceutical compositions, and use of this combination in the treatment of intermittent claudication.

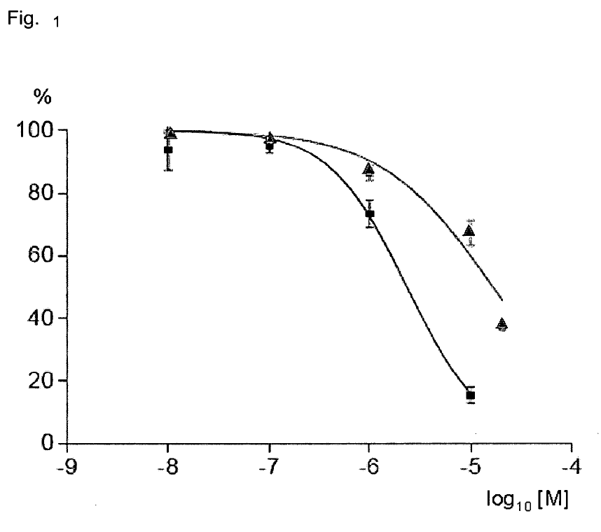

### FIELD OF THE INVENTION

The invention relates to a combination of picotamide (N,N′-bis-(3-picolyl)-4-methoxy-isophthalamide) and naftidrofuryl(nafronyl, 2-diethylaminoethyl 2-(naphthalen-1-ylmethyl)-3-(oxolan-2-yl)propanoate), pharmaceutical compositions containing these, and use of this combination in the treatment of intermittent claudication.

### BACKGROUND OF THE INVENTION

It was described already 30 years ago in French Patent 2 100 850 that N,N′-bis-(3-picolyl)-4-methoxyisophthalamide, hereinafter referred to by its international non-proprietary name “picotamide”, has a high fibrinolytic and anticoagulant activity. The good platelet anti-aggregant activity was disclosed in U.S. Pat. No. 3,973,026. Picotamide is a dual acting thromboxane A2 (TXA2) antagonist and thromboxane synthase inhibitor, and is an effective inhibitor of platelet aggregation and vascular constriction (Gresele et al., Thromb. Haemost. 61:479-84, 1989; Cattaneo et al., Thromb. Res. 62:717-24, 1991).

Likewise it is known for many years that 2-diethylaminoethyl 2-(naphthalen-1-ylmethyl)-3-(oxolan-2-yl)propanoate (hereafter referred to by its non-proprietary names “naftidrofuryl” or “nafronyl”) is an effective medication for treatment of intermittent claudication in patients with peripheral artery disease Fontaine Stage II (De Backer et al., J. Clin. Pharmacol. 56:199-206, 2000; Kieffer et al., Internat. Angiol. 20:58-65, 2001; Goldsmith and Wellington, Drugs Aging 22:967-977, 2005). Nafronyl acts as a serotonin receptor antagonist, but also is able to improve oxygen utilisation and ATP formation in skeletal muscle at the mitochondrial level (Michiels et al., J. Pharmacol. Exp. Ther. 267:904-911, 1993; Mouren et al., Vascular Medicine 3:9-14, 1998). The compound and its antispasmodic properties were described more than 40 years ago in French Patent 3843M, see also U.S. Pat. No. 3,334,096. The peripheral and coronary vasodilatory action and the anaestetic activity were also described. The preferred form of nafronyl is the addition salt with oxalic acid, also known under the trade names Dusodril, Gevatran and Praxilene. It has been described that nafronyl is an agent for the treatment of arterial diseases in limbs, circulatory disorders in the hands and feet, cerebral vascular disorders and diffuse circulatory insufficiency. Nafronyl is the compound of choice for the treatment of intermittent claudication.

The effects of these two drugs by themselves, although deemed clinically significant, are limited, and therefore a medical need exists for more effective treatment modalities for patients suffering from intermittent claudication.

### SUMMARY OF THE INVENTION

The invention relates to a pharmaceutical composition comprising both picotamide and nafronyl, to the preparation thereof, to the use of a combination of picotamide and nafronyl for the treatment of intermittent claudication and other cardiovascular and related diseases, and to a method of treatment of intermittent claudication and other cardiovascular and related diseases using this combination.

### BRIEF DESCRIPTION OF THE FIGURE

Relaxation effect of picotamide alone (-▴-) and nafronyl 1 μM/picotamide mixtures (-▪-) on rat aorta rings pretreated with 8-iso-prostaglandin F2α.

X-axis: Picotamide concentration (log₁₀ [M]). Y-axis: Tension (%).

### DETAILED DESCRIPTION OF THE INVENTION

The mechanisms by which picotamide and nafronyl exert their related, clinically valuable effects are different: Picotamide is a dual acting TXA2 blocker, whereas nafronyl acts as a serotonin receptor antagonist. Because of this non-overlapping mode of actions, the two drugs are possible candidates for a combination treatment. It has now been shown that a mixture of picotamide and nafronyl is more effective than these drugs alone.

The aimed-for goal in preventing or treating muscle cramps is to induce an effective vasorelaxation, enabling sufficient blood flow to muscle tissues to provide for their oxygen and energy substrate needs. The tests below describe the vasorelaxant effect of picotamide and nafronyl alone or in combination as assessed in organ bath experiments with rat aortic rings. Surprisingly, the potency of picotamide (as expressed by the EC₅₀ of the drug in the experiment described below) was strongly enhanced when tested in the presence of nafronyl in a concentration which in itself had little effect, such that the effect of the two drugs together exceeded the sum of the individual drug effects.

The described test is indicative of the activity of the active ingredient or the combination of active ingredients for the treatment of diseases wherein vasorelaxation is expected to cause relief. In particular, the described test is a suitable model for compounds active in the treatment of intermittent claudication.

Picotamide is the international non-proprietary name for N,N′-bis-(3-picolyl)-4-methoxy-isophthalamide of the formula

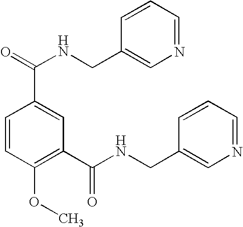

The anhydrous form of picotamide as described in French Patent 2 100 850 has a melting point of 124° C. A crystal form of picotamide monohydrate has been described in UK patent 2 080 288 as having a melting point of 95-97° C.

A further useful form of picotamide are acid salts with inorganic or organic acids. Suitable inorganic acids are, for example, halogen acids, such as hydrochloric or hydrobromic acid, sulfuric acid, or phosphoric acid. Suitable organic acids are, for example, carboxylic, phosphonic, sulfonic or sulfamic acids, for example formic acid, acetic acid, propionic acid, octanoic acid, decanoic acid, dodecanoic acid, glycolic acid, lactic acid, fumaric acid, oxalic acid, malonic acid, succinic acid, adipic acid, pimelic acid, suberic acid, azelaic acid, malic acid, maleic acid, hydroxymaleic acid, tartaric acid, citric acid, amino acids, such as glutamic acid or aspartic acid, methylmaleic acid, cyclohexanecarboxylic acid, adamantanecarboxylic acid, benzoic acid, salicylic acid, 4-aminosalicylic acid, phthalic acid, phenylacetic acid, mandelic acid, cinnamic acid, trifluoroacetic acid, methane- or ethane-sulfonic acid, 2-hydroxyethanesulfonic acid, ethane-1,2-disulfonic acid, 10-camphorsulphonic acid, benzenesulfonic acid, 2-naphthalenesufonic acid, 1,5-naphthalene-disulfonic acid, o-, m- or p-toluenesulfonic acid, methylsulfuric acid, ethylsulfuric acid, dodecylsulfuric acid, N-cyclohexylsulfamic acid, N-methyl-, N-ethyl- or N-propyl-sulfamic acid, or other organic protonic acids, such as ascorbic acid.

Particularly useful are picotamide hydrochloride, hydrobromide, sulphate, phosphate, oxalate, maleate, trifluoroacetate, mesylate(methanesulphonate), p-toluenesulphonate, or 10-camphorsulphonate. Primarily considered are picotamide salts formed with hydrochloric acid (i.e. the hydrochloride) or with sulfonic acids, e.g. with methanesulfonic acid (i.e. the mesylate).

Picotamide, in anhydrous form, as the hydrate or as a salt, is an effective inhibitor of platelet aggregation and vascular constriction, improves the walking distance of patients with peripheral artery disease, is effective in secondary prevention of transient ischemic attacks and stroke (superior to aspirin), is effective in secondary prevention of cardiovascular events in diabetic patients with peripheral artery disease or carotid atherosclerosis, is effective in reducing anginal events in patients with unstable angina or effort angina (where aspirin has no effect), is effective in reducing albuminuria in patients with micro-albuminuria, reduces progression of plaques in carotid atherosclerosis, reduces aura in migraine patients, reduces serum creatinine and pulmonary pressure in congestive heart failure patients, and is useful in related cardiovascular problems.

Nafronyl is the international non-proprietary name for 2-diethylaminoethyl 2-(naphthalen-1-ylmethyl)-3-(oxolan-2-yl)propanoate of the formula

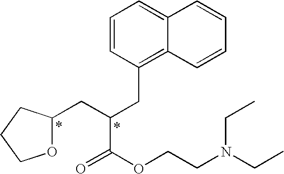

Nafronyl may be used as the free base, but the form most often used in currently marketed medicines is the addition salt with oxalic acid. Other salts known are the fumarate, phosphate, and citrate.

Since position 2 of the propionic acid backbone and position 2 of the tetrahydrofuryl carbon atom in nafronyl are chiral carbon atoms (indicated with an asterix), the compound exists in enantiomeric and diastereomeric forms, as described in EP 0 069 013. Nafronyl when used hereinafter comprises any of these enantiomers 2-diethylaminoethyl 2(R)-(naphthalen-1-ylmethyl)-3-(oxolan-2(R)-yl)propanoate, 2-diethylaminoethyl 2(R)-(naphthalen-1-ylmethyl)-3-(oxolan-2(S)-yl)propanoate, 2-diethylaminoethyl 2(S)-(naphthalen-1-ylmethyl)-3-(oxolan-2(R)-yl)propanoate, and 2-diethylaminoethyl 2(S)-(naphthalen-1-ylmethyl)-3-(oxolan-2(S)-yl)propanoate, and diastereomeric pairs 2-diethyl-aminoethyl 2(R*)-(naphthalen-1-ylmethyl)-3-(oxolan-2(R*)-yl)propanoate and 2-diethyl-aminoethyl 2(R*)-(naphthalen-1-ylmethyl)-3-(oxolan-2(S*)-yl)propanoate, or mixtures thereof. The four enantiomers (and the two diastereomeric racemates) are obtained according to D. Descours et al., Helv. Chim. Acta 74, 1757-1763 (1991) or, preferably, by preparative column chromatography on CHIRALPAK® (Daicel chiral columns for high performance liquid chromatography, Chiral Technologies Europe, F-67404 Illkirch, France).

The present invention relates to pharmaceutical compositions that comprise picotamide, in anhydrous form, as the hydrate or as an acid addition salt, and nafronyl, as free base or as an acid addition salt, as active ingredients and that can be used especially in the treatment of the diseases mentioned hereinbefore, in particular intermittent claudication. Compositions for enteral administration, such as nasal, buccal, rectal or, especially, oral administration, are preferred. The compositions comprise the active ingredients alone or, preferably, together with a pharmaceutically acceptable carrier. The dosage of the active ingredients depends upon the disease to be treated and upon the species, its age, weight, and individual condition, the individual pharmacokinetic data, and the mode of administration.

Whenever the term picotamide is used, this term encompasses any form of picotamide, in particular the anhydrous form, the monohydrate or an acid addition salt, but also other forms, e.g. other crystal forms, hydrates or solvates. Whenever the term nafronyl is used, this term encompasses any form of nafronyl, an enantiomer or diastereomer or mixtures thereof, the free base or acid addition salts, in particular the oxalate, but also other salts, hydrates and solvates, and particular crystal forms.

Within the pharmaceutical composition comprising picotamide and nafronyl as described hereinbefore, picotamide can also be applied as a picotamide salt on a solid pharmaceutically acceptable carrier, preferably a solid carrier composed of carbohydrate units. Such carriers are, for example, sugars, such as mannose, lactose, fructose, glucose, sucrose or saccharose, sugar alcohols, such as mannitol, xylitol or sorbitol, starches, for example corn, wheat, rice or potato starch, cellulose preparations, for example microcrystalline cellulose, methylcellulose, hydroxypropylcellulose(hyprolose), hydroxypropyl methylcellulose(hypromellose), or sodium carboxymethylcellulose, guar gum, carrageenan, or acacia gum. Further solid carriers considered are magnesium or calcium phosphates, for example tricalcium phosphate or calcium hydrogen phosphate, silicium dioxide, silicates, for example magnesium aluminium silicate or calcium silicate, and titanium dioxide. Preferred carriers are microcrystalline cellulose, and, in particular, hydroxymethylpropylcellulose (HMPC) and sodium carboxymethylcellulose.

Suitable additional carriers are especially fillers, such as the sugars, sugar alcohols, cellulose preparations and/or phosphates and silicates mentioned above as carriers, silicium dioxide, and titanium dioxide, and also binders, such as starches, for example corn, wheat, rice or potato starch, guar gum, gelatin, methylcellulose, hydroxypropyl methylcellulose, sodium carboxymethylcellulose, shellac, traganth, xanthan or polyvinyl-pyrrolidone, and/or disintegrators, such as the mentioned starches, also sodium or calcium carboxymethyl starch and sodium glycolate starch, crosslinked polyvinyl-pyrrolidone(crospovidon), croscarmellose, alginic acid or a salt thereof, such as sodium alginate, and colloidal silicium dioxide. Additional excipients are especially flow conditioners and lubricants, for example silicic acid, talc, stearic acid or salts thereof, such as magnesium, zinc or calcium stearate, glycerol monostearate, glycerol palmitostearate, and/or polyethylene glycol, or derivatives thereof.

Dyes or pigments may be added to the tablets, granules, lozenges or chewing-gums, for example for identification purposes or to indicate different doses of the active ingredients.

Pharmaceutical compositions for oral administration also include hard capsules consisting of gelatin, and also soft, sealed capsules consisting of gelatin and a plasticizer, such as glycerol or sorbitol. The capsules may contain the active ingredients in the form of granules, for example in admixture with fillers, such as corn starch, binders, and/or glidants, such as talc or magnesium stearate, and optionally stabilizers.

Pharmaceutical compositions for oral administration also include retard forms, such as retard tablets or capsules, film tablets, and enteric coated tablets. Such special pharmaceutical oral compositions are those according to standard procedures in the art. Retard tablets may, for example, comprise polymeric components such as poly-methacrylates and polymethacrylate copolymers, poylacrylate-polymethacrylate copolymers or related resinuous polymers. Film tablets are obtained by coating with, for example, ethyl cellulose and hydroxypropyl methylcellulose. Suitable enteric coatings are, for example, ethyl cellulose, cellulose acetate phthalate, shellac, hydroxypropyl cellulose acetate succinate, and polymethacrylates and polymethacrylate copolymers.

Pharmaceutical compositions suitable for rectal administration are, for example, suppositories that consist of a combination of the active ingredients and a suppository base. Suitable suppository bases are, for example, natural or synthetic triglycerides, paraffin hydrocarbons, polyethylene glycols or higher alkanols.

The invention relates also to pharmaceutical compositions comprising picotamide and nafronyl for use in a method for the prophylactic or especially therapeutic management of the human or animal body, in particular in a method of treating cardiovascular and related diseases mentioned above, such as intermittent claudication.

The invention relates also to processes for the preparation of pharmaceutical compositions comprising picotamide and nafronyl.

The pharmaceutical compositions comprise from approximately 1% to approximately 50% picotamide, preferably between 10% and 25% picotamide, and from approximately 1% to approximately 50% nafronyl, preferably between 10% and 25% nafronyl. The ratio of picotamide to nafronyl is between 1 to 10 and 10 to 1, preferably between 1 to 2 and 5 to 1, such as between 1 to 1 and 3 to 1, e.g. around 2 to 1, per weight.

Nafronyl free base, as a mixture of diastereomers, is usually an oil. Likewise, all four enantiomers are oils, and also mixtures thereof. Mixtures of nafronyl free base and picotamide as the anhydride or monohydrate in the preferred weight ratios are solids which can be stored for an extended period of time and can easily be handled in solid, pulverized form.

Unit dose forms are, for example, tablets, mini-tablets, granules, capsules containing mini-tablets or granules, lozenges or chewing-gums.

The pharmaceutical compositions of the present invention are prepared in a manner known per se, for example by means of conventional mixing, granulating, dissolving or lyophilizing processes.

Pharmaceutical compositions for oral administration can be obtained, for example, by mixing one of the active ingredients picotamide or nafronyl in solid form with a solid carrier or carrier mixture, adding the other of the two active ingredients in solid form, and, if desired or necessary, adding additional excipients, granulating or tabletting the resulting mixture and optionally filling granules or mini-tablets into capsules, or adding the mixture to a suitable material for the preparation of lozenges or chewing-gums. Alternatively, one or both active ingredient may be used in solution, carriers and additional excipients added to this solution, the solution evaporated, and the resulting solid granulated or tabletted.

Preferred pharmaceutical compositions for oral administration comprising nafronyl and picotamide as acid addition salts can be obtained, for example, by mixing picotamide (free base) with a solution of the acid for salt formation and with one or more solid carriers, adding nafronyl (as acid addition salt or as free base) in solid form, if desired or necessary, adding additional excipients, evaporating the solvent and further processing as described above.

Pharmaceutical compositions of the invention comprising a picotamide salt and a nafronyl salt as described hereinbefore dissolve readily. Dissolution already occurs in the mouth with saliva, allowing buccal absorption, with a rapid onset of the desired activity of the active ingredient. This is particularly important, if patients with angina attacks or claudication pain want relief as quickly as possible. Buccal absorption is also observed in pharmaceutical compositions in the form of lozenges containing soft gum. Such lozenges may be particularly important for patients having difficulties in chewing and/or swallowing tablets.

If it is desirable to have an extended activity of the mixture of the active ingredients of the invention, the preferred pharmaceutical composition is a slow release formulation, e.g. a film tablet or other slow release form comprising methacrylate copolymers as described above.

The present invention relates furthermore to a method for treatment of cardiovascular or related diseases, in particular intermittent claudication, which comprises administering a mixture of picotamide and nafronyl in a quantity effective against said disease, to a warm-blooded animal requiring such treatment. The mixture of picotamide and nafronyl can be administered as such or especially in the form of pharmaceutical compositions, prophylactically or therapeutically, preferably in an amount effective against the said diseases, to a warm-blooded animal, for example a human, requiring such treatment. In the case of an individual having a bodyweight of about 70 kg the daily dose administered is from approximately 0.05 g to approximately 5 g, preferably from approximately 0.25 g to approximately 1.5 g, such as between 0.4 g and 1.0 g, of a mixture comprising picotamide and nafronyl, especially a mixture with a preferred ratio of picotamide to nafronyl between 5 to 1 and 1 to 2.

The present invention relates also to the use of a mixture of picotamide and nafronyl, especially those mentioned as being preferred, as such or in the form of a pharmaceutical formulation with at least one pharmaceutically acceptable carrier for the therapeutic and also prophylactic management of one or more of the diseases mentioned hereinabove, in particular cardiovascular diseases, e.g. intermittent claudication. The invention furthermore relates to the use of a mixture of picotamide and nafronyl, especially in the ratio mentioned above as being preferred, for the manufacture of pharmaceutical compositions for the treatment of cardiovascular and related diseases.

### EXAMPLES

The following Examples serve to illustrate the invention without limiting the invention in its scope.

### Example 1

### Stability of Picotamide-Nafronyl Combination in Solid State Form

Manufacture of Picotamide-Nafronyl 2:1 Mixture

1 g Nafronyl oxalate (Sigma Aldrich N1391-6G) is suspended in 10 ml dichloromethane and cooled to 0-5° C. 10 ml water and 3 ml of a 1 M sodium hydroxide solution are added. The mixture is shaken vigorously and the organic phase is washed several times with water until the pH value of the water solution is neutral. The organic phase is dried and the solvent removed by evaporation, resulting in 748.7 mg of the free base as a light yellow oil. HPLC analysis gives no evidence of formation of by-products.

748.7 mg Nafronyl free base and 1469.7 mg picotamide anhydride (Sai Advantium Pharma Ltd.) are dissolved in 30 ml ethanol. The solvent is removed by evaporation at 40° C. and reduced pressure, and the residue is dried in a desiccator under reduced pressure, resulting in 2.239 g solidified foam.

Storage Stability of Picotamide-Nafronyl 2:1 Mixture

40 mg Picotamide-nafronyl 2:1 mixture is placed into a 96 well plate format glass vial equipped with a humidity control salt solution container (saturated NaCl for 40° C./75% relative humidity and saturated NaBr for 25° C./60% relative humidity). The glass vial is hermetically sealed and the test samples are stored at the desired temperature on vibrational spectroscopic screening system SpecScreen xHTS (RPD TOOL AG). During the storage near infrared (NIR) and Raman spectra are automatically acquired at predetermined time intervals. The spectra are compared with spectra of nafronyl (free base), picotamide anhydride and picotamide monohydrate (European Pharmacopoeia), and purity tested with HPLC on a Nucleosil 100 C18 column, solvent gradient acetonitrile 1 % to 60% in 0.1% aqueous phosphoric acid.

The mixture picotamide-nafronyl 2:1 (by weight) upon 6 weeks storage at 40° C. and 60% or 75% relative humidity takes up a small amount of water in the first few days to form the picotamide monohydrate, but thereafter does not increase its water content any further, and displays stable solid-state behaviour without any evidence of degradation of the individual components.

### Example 2

### Aortic Relaxation by Picotamide, Nafronyl and Picotamide-Nafronyl Combinations

Design of Test System

Thoracic aortic rings obtained from male Wistar rats are carefully cleaned of connective tissue. The aortic rings are suspended in the isolated muscle bath (20 mL). Each segment is suspended under a tension of 1 g in Krebs buffer (NaCl 118 mM, KCl 5.4 mM, CaCl₂ 2.5 mM, MgCl₂.6H₂O 1.5 mM, NaHCO₃ 25 mM, NaH₂PO₄ 1.2 mM, glucose 10 mM; pH 7.4) which was bubbled with O₂ 95%/CO₂ 5% at 37° C. in 20 mL organ baths (EMKA Technologies, Paris, France).

The muscle tension of aortic rings is isometrically recorded with a force-displacement transducer IT1 (EMKA Technologies, Paris, France). The buffer is renewed at 15 min intervals during the equilibrium period (40 min) before exposing the rings to the TXA2-mimetic U-46619 (20 nM), 8-iso-prostaglandin F2α (1 μM) or serotonin (5-hydroxy-tryptamine, 5-HT, 2 μM). When a stable tension is obtained (15 min), the compound of interest is added at a cumulative increasing concentration to the bath until the tension returned to the baseline value. Tension is recorded with a specific data acquisition program (IOX 1.445, EMKA Technologies, Paris, France). The EC₅₀ value of each drug is assessed for at least 6 concentration-response curves obtained from separate preparations. EC₅₀ is expressed as the concentration which reduces 50% of the tension induced by the contracting agent. The results are expressed as mean ±S.E.M. Nafronyl oxalate was obtained from Sigma-Aldrich; picotamide monohydrate was obtained from the European Directory of Quality of Medicines.

Contracting Effect of U-46619, 8-iso-prostaglandin F2α and Serotonin

EC₅₀ for serotonin was found to be 0.98±0.09 μM, for 8-iso-PGF2α it was 1.0±0.1 μM, and for U-46619 it was 12.16±2.68 nM. These values are in line with results obtained in previous studies (S. W. Watts and J. M. Thompson, J Pharmacol Exp Ther 309:165-172, 2004; M. Lohn et al., FASEB J 16:1057-63, 2002).

Relaxation by Nafronyl, Picotamide and Picotamid-Nafronyl Mixtures Using Serotonin 2 μM as Contracting Agent.

Nafronyl oxalate was confirmed to be a potent serotonin antagonist with an EC₅₀ 1.54·10⁻⁸ M for its relaxing effect. Its potency did not materially change in the presence of 10⁻⁶ M picotamide: EC₅₀ 1.33·10⁻⁸ M. Picotamide monohydrate was a very weak relaxant using serotonin as agonist: tension could only be reduced by 30% with 10⁻⁵ M picotamide.

Relaxation by Picotamide, Nafronyl and Picotamide-Nafronyl Mixtures Using 8-iso-prostaglandin F2α 1 μM as Contracting Agent

Relaxation experiments on aortas precontracted with 8-iso-prostaglandin F2α 1 μM were done with picotamide monohydrate alone in increasing concentrations of 10⁻⁸ M, 10⁻⁷ M, 10⁻⁶ M, 10⁻⁵ M, 10⁻⁴ M, as well as with the same concentrations of picotamide monohydrate in the presence of 1 μM nafronyl. The results are shown in the FIGURE, demonstrating that nafronyl 1 μM induces a left-ward shift of the dose-response curve for picotamide.

Calculated EC₅₀ values:

Picotamide monohydrate alone: EC₅₀ 1.64·10⁻⁵ M

Picotamide monohydrate together with 10⁻⁶ M nafronyl oxalate: EC₅₀ 2.34·10⁻⁶ M

Nafronyl oxalate alone: EC₅₀ 3.88·10⁻⁶ M

1. A pharmaceutical composition comprising picotamide and nafronyl.

2. The composition of claim 1 wherein picotamide is in the form of picotamide anhydride.

3. The composition of claim 1 wherein picotamide is in the form of picotamide monohydrate.

4. The composition of claim 1 wherein picotamide is in the form of a picotamide acid addition salt.

5. The composition of claim 1 wherein nafronyl is in the form of nafronyl free base.

6. The composition of claim 1 wherein nafronyl is in the form of an acid addition salt.

7. The composition of claim 1 wherein nafronyl is in the form of nafronyl oxalate.

8. The composition of claim 5 wherein nafronyl is one of the 4 enantiomers.

9. The composition of claim wherein nafronyl is one of the 2 diastereomeric pairs.

10. The composition of claim 1 comprising between 10% and 25% picotamide and between 10% and 25% nafronyl.

11. The composition of claim 1 wherein the ratio of picotamide to nafronyl is between 1 to 2 and 5 to 1.

12. The composition of claim 1 wherein the ratio of picotamide to nafronyl is between 1 to 1 and 3 to 1.

13. The composition of claim 1 in the unit dose form of tablets, mini-tablets, granules, capsules containing mini-tablets or granules, lozenges or chewing-gums.

14. A method of treatment of intermittent claudication and other cardiovascular and related diseases, which comprises administering a mixture of picotamide and nafronyl in a quantity effective against said disease, to a warm-blooded animal requiring such treatment.

15. The method of claim 14 for the treatment of intermittent claudication.

16. The composition of claim 6 wherein nafronyl is one of the 4 enantiomers.

17. The composition of claim 6 wherein nafronyl is one of the 2 diastereomeric pairs.
